(19) United States
(12) Patent Application Publication
(10) Pub. No.: US 2004/0069311 A1
(43) Pub. Date: Apr. 15, 2004

(54) Medical support system

(75) Inventors: Hajime Sasaki, Kawasaki (JP); Yoshitaka Bito, Tokorozawa (JP); Hideyuki Ban, Hachioji (JP); Yukio Koyanagi, Tokyo (JP)

Correspondence Address: Stanley P. Fisher, Reed Smith Hazel & Thomas LLP, Suite 1400, 3110 Fairview Park Drive, Falls Church, VA, 22042-4503, US

(73) Assignee: Hitachi, Ltd.

(21) Appl. No.: 10/627,673

(22) Filed: Jul. 28, 2003

(30) Foreign Application Priority Data
Oct. 11, 2002 (JP) 2002-298224

(51) Int. Cl. A61B 19/00

(52) U.S. Cl. 128/897

(57) ABSTRACT

A medical support system for storing medical information disclosed by a medical institution in a data management institution is provided. The system comprises patient terminals 10 used by patients 1, medical institution terminals 20 used by medical institutions 2, a management server 30 operated by the data management institution 3, and a network 40 which establishes connections among these elements. The management server 30 has a medical information storage device 32, which stores medical information 4 on the patient such as medical records 41, examination results 42, and medical images 43, and a medical information control device 31 for controlling the input and output of the medical information into and from the medical information storage device. The management server 30 provides medical information management services including services of medical information upload, medical information download, and medical information reference, to patient terminals and medical institution terminals, which are connected via the network, under the patient's permission and authentication. Thus, the patient can control the upload and download of medical information.

### BACKGROUND OF THE INVENTION

1. Technical Field

The present invention relates to a medical support system for exchanging medical information between patients and medical institutions via a network.

2. Background Art

In recent years, there has been an increasing demand for the realization of medical care with high efficiency and high quality through the effective utilization of medical resources by associations among a plurality of medical institutions. However, in general, when a patient receives medical care at a plurality of medical institutions, medical information such as medical records, examination records, or medical images is generated at each medical institution and individually managed thereby. The information management is carried out by various methods such as those using paper or electronic data. Further, at present the electronic data management method differs depending on the medical institutions involved. Accordingly, it is difficult for a medical institution to perform medical care after consistently grasping medical information such as past examination results, or histories of medical treatments which a patient has undertaken in the past. A system has been proposed wherein medical information from a plurality of medical institutions is centrally managed by a database at a data management institution, so that medical information can be commonly used (Patent document 1).

Patent Document 1: JP Patent Publication (Kokai) No. 11-45304 A (1999)

However, the above prior art is problematic in that a patient cannot control how and when his or her own medical information is used, because medical institutions play key roles in managing the sharing of medical information and the like. For example, when a patient wishes a newly visited medical institution to refer to his or her past medical information from a plurality of medical institutions, the patient has to make requests to individual medical institutions. On the other hand, there is a danger that medical institutions would commonly use individual medical information, which is private in nature, without the knowledge of the patient.

### SUMMARY OF THE INVENTION

It is therefore an object of the present invention to provide a medical support system capable of storing medical information on patients disclosed by a plurality of medical institutions in a data management institution and allowing the patients to control the upload and download of the medical information.

In order to achieve the object of the invention, the present invention provides a medical support system comprising patient terminals used individually by a plurality of patients, medical institution terminals used individually by a plurality of medical institutions, and a management server operated by a data management institution, which are connected to each other via a network. The management server comprises a medical information storage device for storing medical information of patients including medical records, examination results, and medical images, and a medical information control device for controlling the input and output of the medical information into and from the medical information storage device. The medical information control device performs an upload request step (1-1), an upload stand-by step (1-2), and an upload execution step (1-3). In step (1-1), the medical information control device transmits upload permission key data for uploading the medical information of the patient to the management server and a medical information upload request content to be registered by a patient's request to the medical institution terminal used by the medical institution, when receiving a request from the patient terminal to upload the medical information of the patient managed by the medical institution to the management server. In step (1-2), the medical information control device waits ready to receive a medical information transmitted from the medical institution terminal, when authenticating the validity of the upload permission key data received from the medical institution terminal. In step (1-3), the medical information control device stores the medical information in the medical information storage device when receiving the medical information transmitted from the medical institution terminal.

Further, the medical information control device performs a download request step (2-1), a download permission step (2-2), and a download execution step (2-3). In step (2-1), the medical information control device transmits the content of the medical information download request to the patient terminal used by the patient, when receiving a medical information download request for viewing the medical information of the patient from the medical institution terminal used by the medical institution. In step (2-2), the medical information control device transmits download permission key data which permits the medical institution to download and view the medical information to the medical institution terminal, when receiving a permission response for downloading the medical information from the patient terminal. In step (2-3), the medical information control device transmits the medical information to the medical institution terminal, when authenticating the validity of the download permission key data received from the medical institution terminal.

Furthermore, the medical information control device performs a reference request step (3-1), a reference permission step (3-2), and a reference execution step (3-3). In step (3-1), the medical information control device transmits the content of the medical information reference request to the patient terminal used by the requested patient, when receiving a medical information reference request from the terminal of a first medical institution to allow a second medical institution, to which the patient is referred to, to download and view the medical information of the patient. In step (3-2), the medical information control device transmits a download permission key data to permit the second medical institution to download and view the medical information to the second medical institution, when receiving a permission response from the patient terminal to permit the second medical institution to download and view the medical information. In step (3-3), the medical information control device transmits the medical information to the second medical institution terminal, when authenticating the validity of the download permission key data received from the second medical institution terminal.

This specification includes part or all of the contents as disclosed in the specification and/or drawings of Japanese Patent Application No. 2002-298224, which is a priority document of the present application.

### BRIEF DESCRIPTION OF THE DRAWINGS

FIG. 1 is a block diagram showing the configuration of a medical support system according to an embodiment of the present invention.

FIG. 2 is a flow chart showing the outline of major service contents provided by the medical support system according to the embodiment of the present invention.

FIG. 3 is a flow chart showing an example of the procedure for uploading medical information according to the embodiment of the present invention.

FIG. 4 is a view showing an example of the display of a medical information upload request page according to the embodiment of the present invention.

FIG. 5 is a view showing an example of the format for storing the upload request data according to the embodiment of the present invention.

FIG. 6 is a view showing an example of the display of a medical information upload page according to the embodiment of the present invention.

FIG. 7 is a view showing an example of the format for storing upload history data according to the embodiment of the present invention.

FIG. 8 is a view showing an example of the format for storing the medical information according to the embodiment of the present invention.

FIG. 9 is a flow chart showing an example of the procedure for downloading medical information according to the embodiment of the present invention.

FIG. 10 is a view showing an example of the display of a medical information download request page according to the embodiment of the present invention.

FIG. 11 is a view showing an example of the format for storing download request data according to the embodiment of the present invention.

FIG. 12 is a view showing an example of the display of a medical information download permission page according to the embodiment of the present invention.

FIG. 13 is a view showing an example of the format for storing download permission data according to the embodiment of the present invention.

FIG. 14 is a view showing an example of the format for storing download history data according to the embodiment of the present invention.

FIG. 15 is a flow chart showing an example of the procedure for medical information reference according to the embodiment of the present invention.

### DETAILED DESCRIPTION OF THE INVENTION

Hereinafter, embodiments of the present invention will be described in detail by referring to the drawings. To simplify the description below, “medical support system” and “medical information management service” will be referred to as “support system” and “management service,” respectively. A “step” will be referred to as “S.”

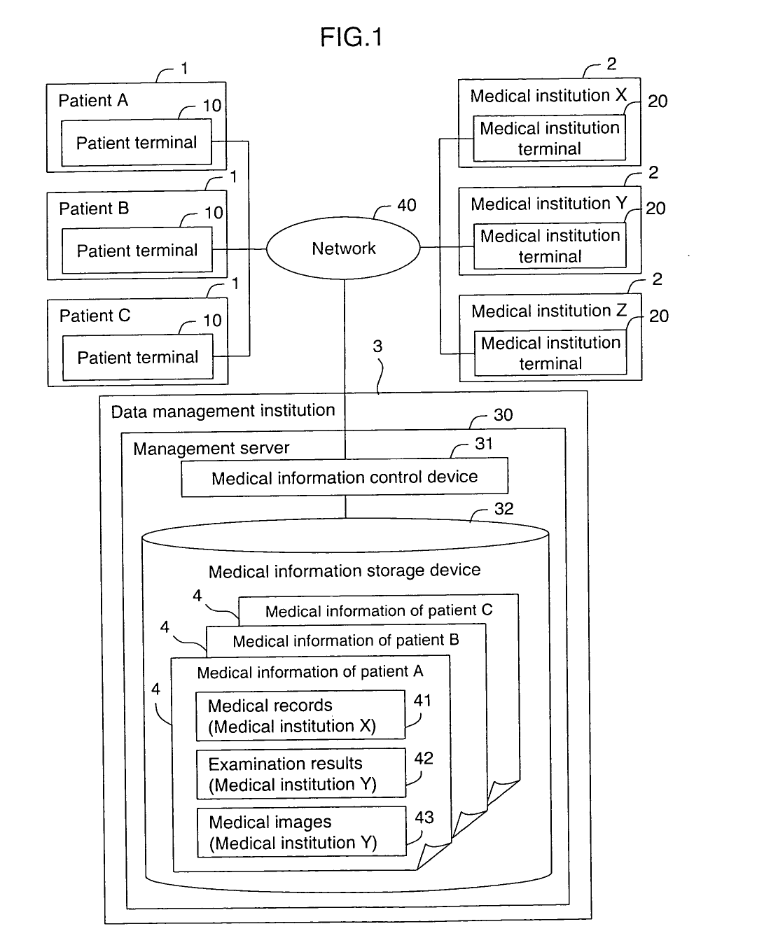

FIG. 1 is a block diagram showing the configuration of a support system according to an embodiment of the present invention. The support system comprises patient terminals 10 used individually by a plurality of patients (A, B, C, . . . ) 1, medical institution terminals 20 used individually by a plurality of medical institutions (X, Y, Z, . . . ) 2, a management server 30 operated by a data management institution 3, and a network 40 through which the above terminals and server are connected to each other.

The patient terminals 10 and the medical institution terminals 20 are information processing apparatuses such as personal computers or handheld terminals, which can be connected to the network 40 for the input and output of information. The management server 30 is an information processing apparatus such as a personal computer, a work station, or a general-purpose computer. The management server 30 has a medical information storage device 32 and a medical information control device 31. The medical information storage device 32 stores medical information 4 such as medical records 41, examination results 42, and medical images 43 concerning a plurality of patients. The medical information control device 31 controls the input and output of the medical information into and from the medical information storage device 32.

The management server 30 provides management services (medical information management services) to the patient terminals 10 and the medical institution terminals 20 connected through the network 40. The management services may be provided by causing the patient terminals 10, medical institution terminals 20, and management server 30 to operate dedicated programs. Alternatively, the management server 30 may manage dedicated management service (medical information management service) Web pages to provide management services which can be connected to the patient terminals 10 and the medical institution terminals 20 via the Internet. Such management services may be combined with e-mails to provide management services. The management server 30 and the management services are basically provided by a service operating company, and patients 1 and medical institutions 2 become members of the operation. Such management services may be provided by a municipal government, union, or insurance company, and the member of the operation may be residents of the municipality, members of union, or clients of insurance company.

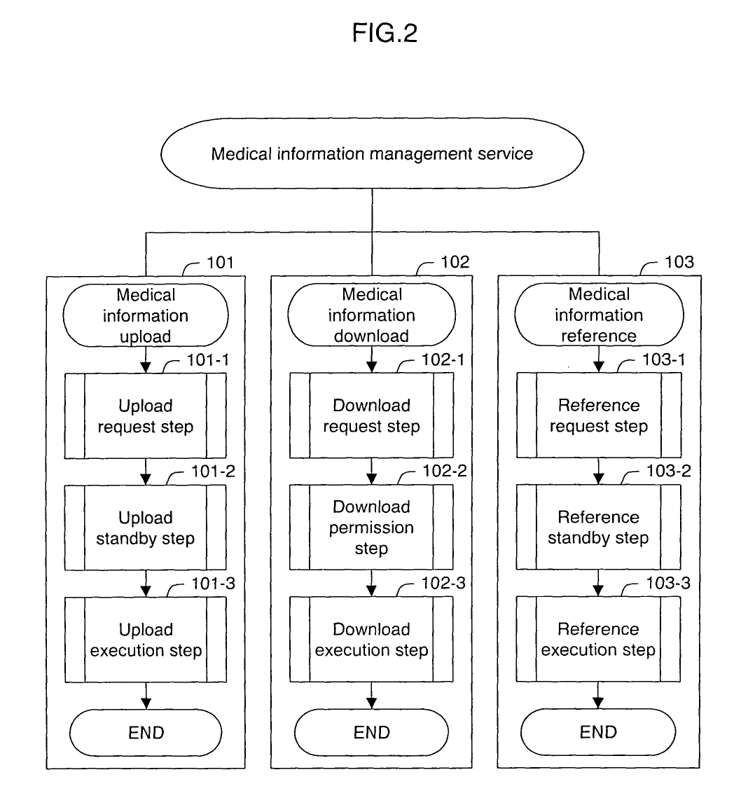

FIG. 2 is a flow chart showing the outline of major service contents of the support system according to the embodiment of the present invention. In this embodiment, the major services include medical information upload 101, medical information download 102, and medical information reference 103.

(1) In the medical information upload 101, a patient 1 requests upload of medical information 4 to a medical institution 2, and an operator of the requested medical institution 2 uploads the medical information 4 of the patient 1 to the medical information storage device 32 of the management server 30. The medical information upload 101 is achieved by upload request S101-1, upload standby S101-2, and upload execution S101-3.

(2) In the medical information download 102, the operator of the medical institution 2 requests permission to download and view the medical information 4 from the patient 1, and with the patient's permission the operator download the medical information 4 of the patient 1. The medical information download 102 is achieved by download request S102-1, download permission S102-2, and download execution S102-3.

(3) In the medical information reference 103, when the operator of a first medical institution (X) 2 refers the patient 1 to a second medical institution (Y) 2, the operator of the first medical institution requests the patient 1 to allow the second medical institution 2 to download and view the medical information 4. With the patient's permission an operator of the second medical institution 2 downloads the medical information 4. The medical information reference 103 is achieved by reference request S103-1, reference stand-by S103-2, and reference execution S103-3.

(Description of the Procedure of Medical Information Upload 101)

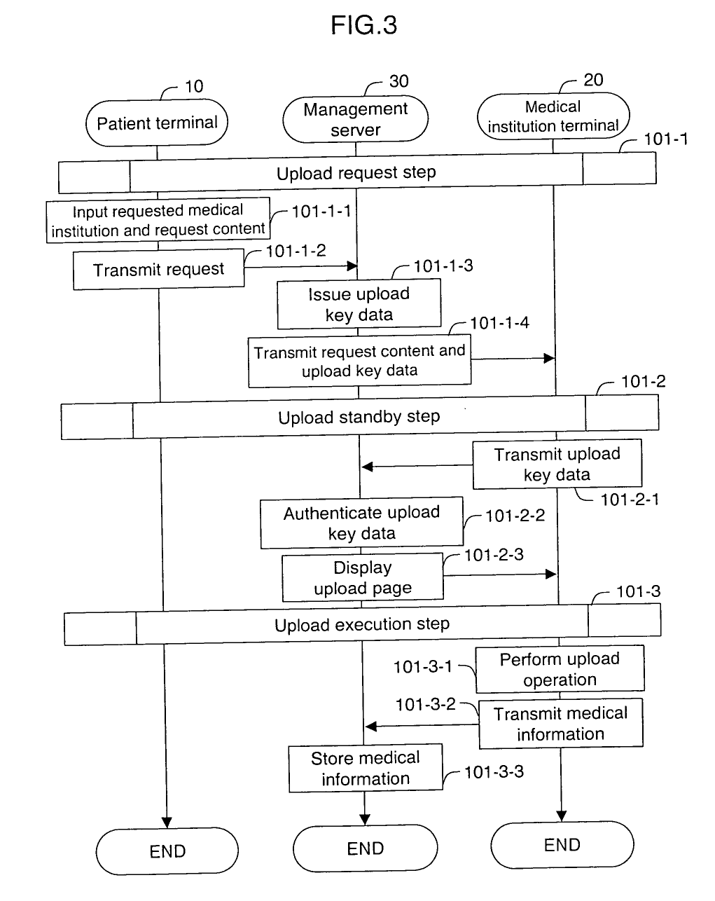

FIG. 3 is a flow chart showing an example of the procedure of the medical information upload 101 according to the embodiment of the present invention. When the patient 1 requests a medical institution 2 to upload medical information 4, the support system first executes the upload request S101-1. The patient 1 inputs the name of the requested medical institution 2 and the request content through the patient's terminal 10 (S101-1-1) and the request is transmitted to the management server 30 (S 101-1-2).

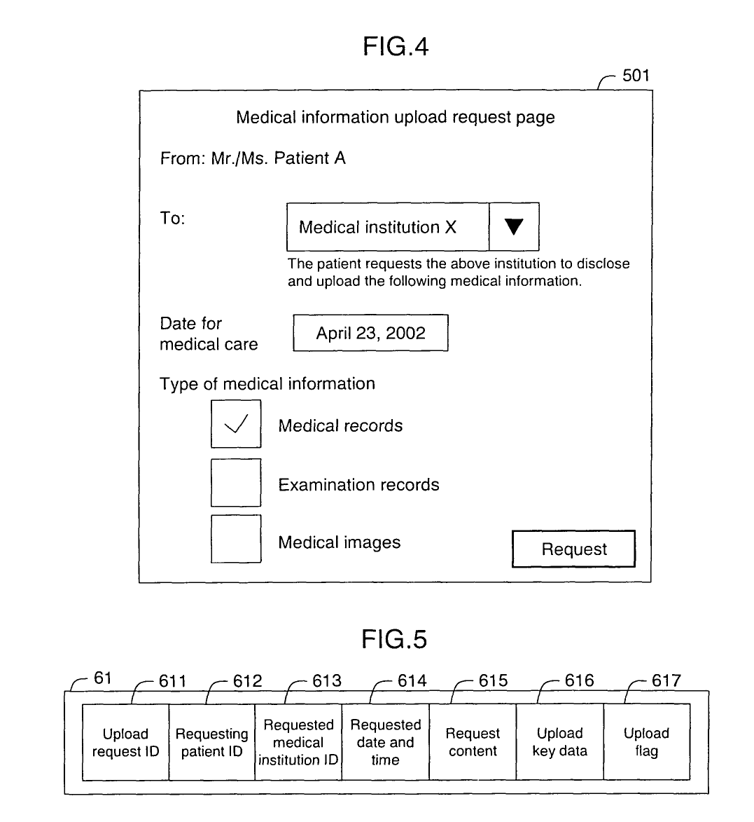

FIG. 4 is a view showing an example of the display of a medical information upload request page 501 according to the embodiment of the present invention. In the example shown in FIG. 4, a medical institution 2 (herein referred to as “X”), which is requested to upload the medical information, the date and time of medical care which is desired to be uploaded (which is Apr. 23, 2002 in the example), and the type of medical information 4 which is desired to be uploaded (which is “medical records” in the example) are inputted on the page. When the patient 1 clicks an icon indicating “REQUEST,” the request is transmitted to the management server 30. Next, after receiving the request, the management server 30 issues upload key data necessary for the upload of the medical information 4 (S101-1-3) and transmits the request content and the upload key data to a medical institution terminal 20 operated by an operator of the requested medical institution 2 (S101-1-4). Then, the management server 30 generates and stores upload request data including the upload key data.

FIG. 5 is a view showing an example of the format for storing the upload request data according to the embodiment of the present invention. The upload request data 61 includes an upload request ID 611, a requesting patient ID 612, a requested medical institution ID 613, a requested date and time 614, a request content 615, upload key data 616, and an upload flag 617.

The upload key data is created as a different string of data for each upload request. The upload key data may be a character string created by converting by hash function from a character string including the patient ID of the requesting patient 1, the medical institution ID of the requested medical institution 2, and the request content. When the request is made through the page 501 shown in the example in FIG. 4, the request content includes information such as the date and time of medical care and type of medical information. All of such request contents may be stored as text data. The items of the registration request data 61 may be defined in more detail than the example shown in FIG. 5 by, for example, storing the date and time of medical care and the type of medical care separately.

At S101-1-4, the request contents and upload key data may be transmitted by e-mail to the terminal 20 of the requested medical institution, or transmitted through operations on the Web page accessed from the medical institution terminal 20. This is also true for transmission processes described below.

While an operator of the medical institution 2 having received the request prepares for the upload of the medical information 4, the support system executes the upload stand-by S101-2. First, the operator of the medical institution 2 transmits the upload key data from the medical institution terminal 20 to the management server 30 (S101-2-1). For example, this can be conducted by having the management server 30 add the address of the Web page for accepting the upload key data to the e-mail transmitted, so that the upload key data can be transmitted through operations on the Web page accessed from the medical institution terminal 20. This is also true for key data transmission processes described below.

Next, the management server 30 confirms whether or not the received upload key data and the ID of the transmitting medical institution are present in the upload requested data 61. It then authenticates the upload key data (S101-2-2), and displays an upload page to upload the medical information 4 (S101-2-3).

The support system performs upload execution S101-3 until the operator of the requested medical institution 2 has prepared for the upload of the medical information 4 to the management server 30 and then has completed the upload. First, medical information to be uploaded is inputted or designated through a medical information upload page displayed on the screen of the medical institution terminal 20 (S101-3-1), and then the medical information is transmitted to the management server 30 (S101-3-2).

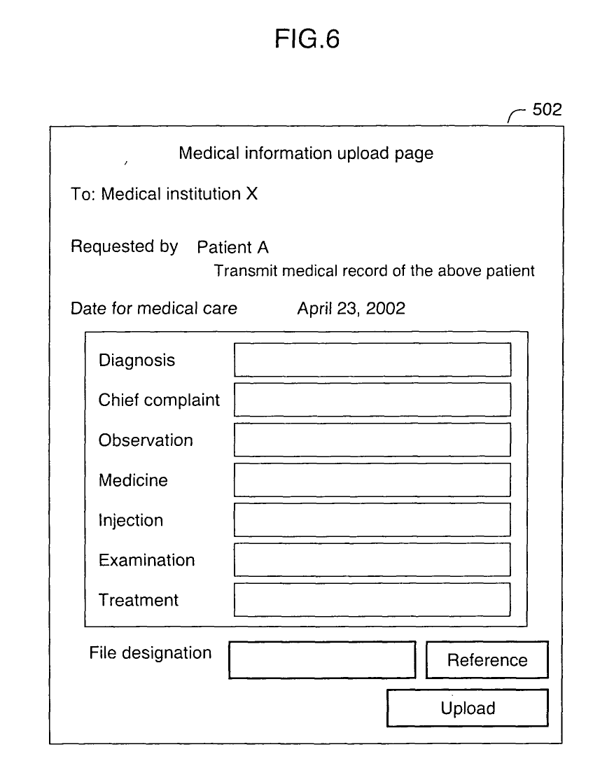

FIG. 6 is a view showing an example of the display of the medical information upload page 502 according to the embodiment of the present invention. In the example of FIG. 6, an input page for a medical record is illustrated. When, for example, the upload of an examination record is requested, a separate upload page may be displayed or a page capable of uploading both records at the same time may be displayed. In the example shown in FIG. 6, the name of a medical institution (here described as “X”), the name of a patient requesting the upload (here described as “A”), and the date and time of medical care whose information is requested to be uploaded (here described as “Apr. 23, 2002”) are displayed. At the same time, input boxes are displayed for inputting the diagnosis, chief complaints, observations, medicines, injection, examinations, and treatments.

In this example of format, the input is made by entering character information into input boxes by a keyboard. The input may be made by selecting from a list displayed on the page or by entering an image of a memo of handwritten line-work. Further, for example, the files of the medical information 4 stored as XML may be designated on the medical information upload page 502 based on the medical information 4 stored independently by the medical institution 2.

When the operator of the medical institution 2 clicks an icon “UPLOAD,” the medical information is transmitted. After receiving the medical information, the management server 30 stores the medical information (S101-3-3) and then the series of processes in medical information upload 101 ends. When storing the medical information, the management server 30 modifies an upload flag 617 in the corresponding upload requested data 61 from “not uploaded” to “uploaded,” and at the same time creates and stores upload history data and medical information data.

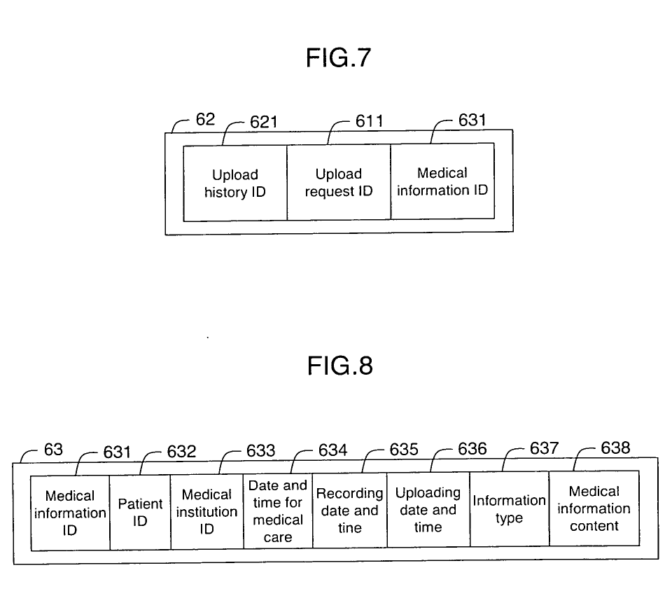

FIG. 7 is a view showing an example of the format for storing the upload history data according to the embodiment of the present invention. The upload history data 62 includes an upload history ID 621, an upload request ID 611, and a medical information ID 631. When a plurality of medical information items are present relative to one upload request, a plurality of upload history data 62 having the same upload request ID 611 are stored.

FIG. 8 is a view showing an example of the format for storing the medical information according to the embodiment of the present invention. The medical information 63 includes a medical information ID 631, a patient ID 632, a medical institution ID 633, a date and time of medical care 634, a recording date and time 635, an uploading date and time 636, an information type 637, and medical information content 638. As the information type 637, types of medical information such as medical records, examination records, and medical images are stored. The medical information content 638 may employ such a format that the transmitted medical information is stored in XML format or an image data format, or a pointer toward a file or the like which is separately stored is stored.

As is clear from the above description, according to the embodiment of the present invention, it is possible for a patient to determine when and by whom the patient's medical information is stored, and which medical information is to be stored, so that the patient can safely control the information. Since the medical information is collected by a patient from information disclosed by medical institutions, the patient can freely utilize for themselves or allow medical institutions to view it.

(Description of the Procedure of Medical Information Download 102)

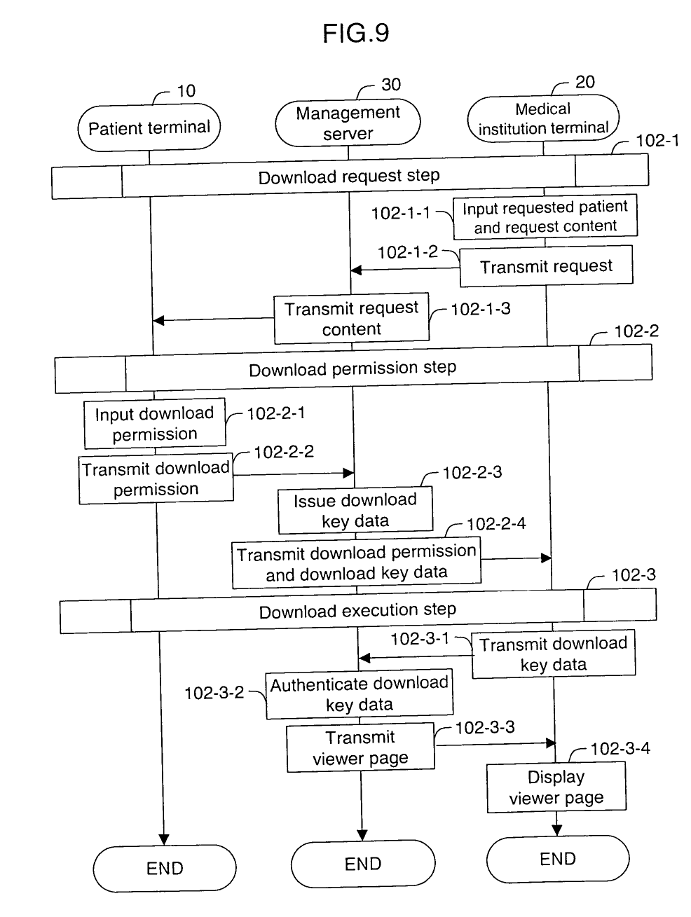

FIG. 9 is a flow chart showing an example of the procedure of the medical information download 102 according to the embodiment of the present invention. When the operator of the medical institution 2 requests the patient 1 to allow the medical institution 2 to view his past medical information 4, the support system first performs download request S102-1. The operator of the medical institution 2 inputs the name of requested patient 1 and the content of the request on the medical institution terminal 20 (S102-1-1), and then transmits the request to the management server 30 (S102-1-2).

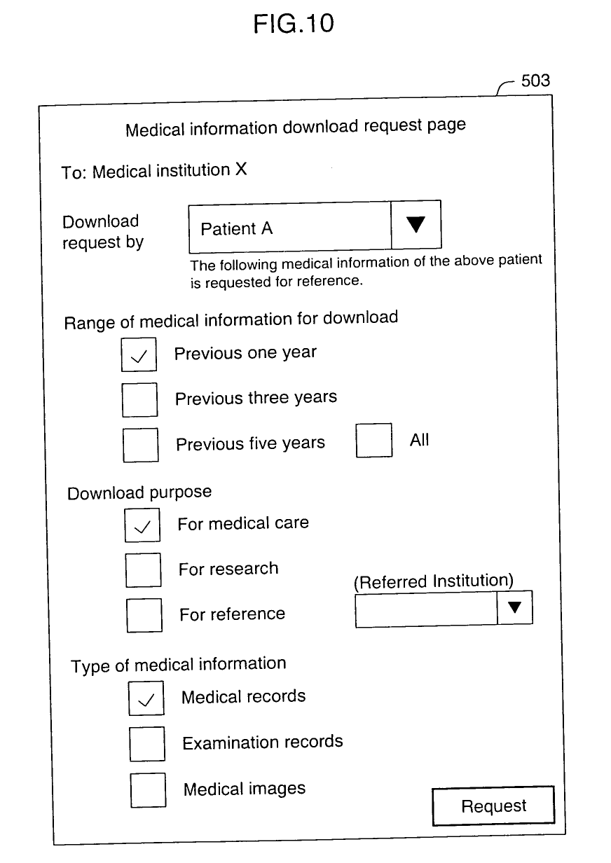

FIG. 10 is a view showing an example of the display of a medical information download request page 503 according to the embodiment of the present invention. In the example shown in FIG. 10, the name of the requested patient 1 (here described as “A”), the range of medical information, that is how many years of the past medical information is to be downloaded (here described as “previous one year”), the purpose of download (here described as “for medical care”), and the type of medical information to be downloaded (here described as “medical records”) are inputted on the page. When the operator of the medical institution 2 clicks an icon indicating “REQUEST,” the request is transmitted to the management server 30. Next, upon the reception of the request, the management server 30 transmits the request content to the patient's terminal 10 used by the requested patient 1 (S102-1-3). At that time, the management server 30 creates and stores download request data.

FIG. 11 is a view showing an example of the format for storing download request data according to the embodiment of the present invention. The download request data 64 includes a download request ID 641, a requesting medical institution ID 642, a requested patient ID 643, a request date and time 644, a request content 645, and a permission flag 646. The request content 645 may store the entire content of the request as text data. The items of the download request data 64 may be defined in greater detail than the example shown in FIG. 11, such that the range of medical information, the reference purpose, and the medical information type are stored as separate items.

When the patient 1 having received the request gives permission for downloading the medical information, the support system performs download permission S102-2. First, the patient 1 confirms the contents of the download request by e-mail or on the Web page using the patient terminal 10, and then inputs download permission (S102-2-1). As a result, the patient terminal 10 transmits permission for medical information download to the management server 30 (S102-2-2).

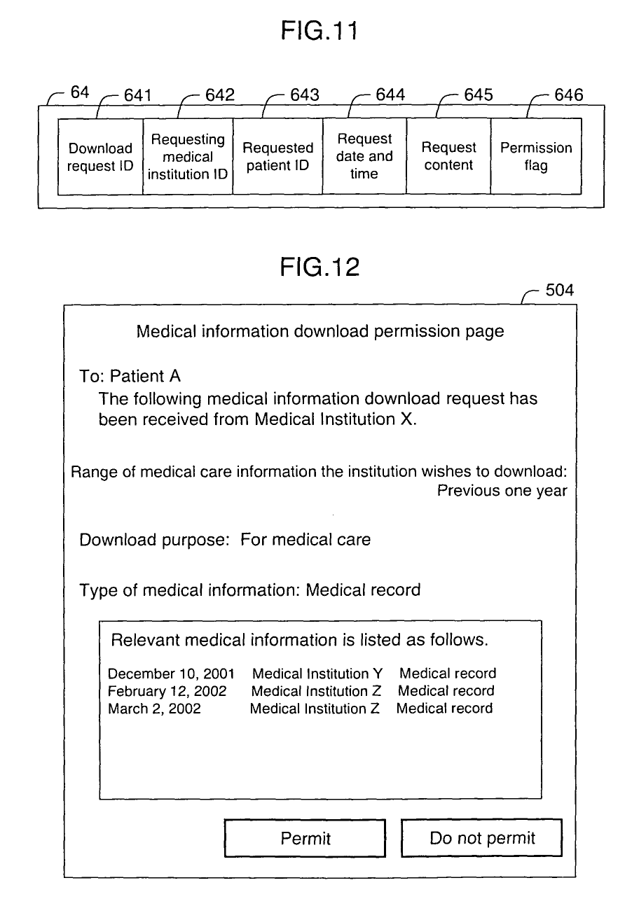

FIG. 12 is a view showing an example of the display of a medical information download permission page 504 according to the embodiment of the present invention. In the example shown in FIG. 12, the contents of the download request from a medical institution (X), that is the range (period) of information of the download request, the download purpose, and a list of target medical information, are displayed, and the patient can confirm them. When the patient 1 (A) confirms the displayed contents of the page 504 shown in FIG. 12 and clicks an icon indicating “PERMIT,” S102-2-2 is performed. The management server 30 having received the download permission issues download key data necessary for the download of the medical information 4 (S102-2-3), and then transmits the download permission and download key data to the medical institution terminal 20 used by the operator of the requesting medical institution 2 (S102-2-4). At the same time, the management server 30 modifies a permission flag 646 in the corresponding download request data 64 from “not permitted” to “permitted,” and creates and stores download permission including download key data.

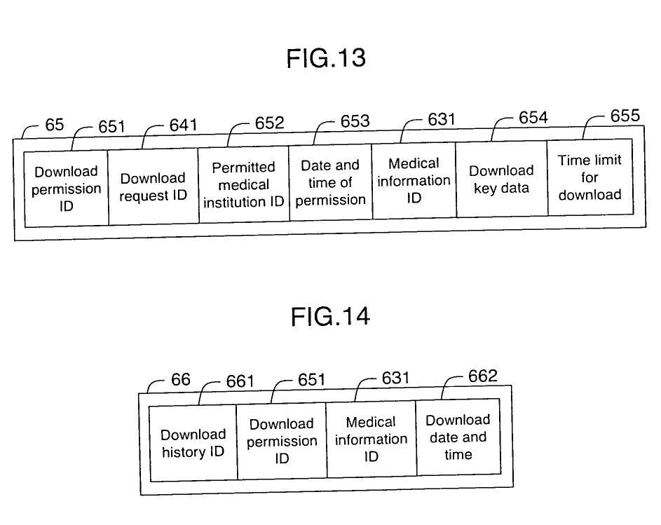

FIG. 13 is a view showing an example of the format for storing download permission data according to the embodiment of the present invention. The download permission data 65 includes a download permission ID 651, a download request ID 641, a permitted medical institution ID 652, a date and time of permission 653, a medical information ID 631, download key data 654, and a time limit for download 655. When there are plural medical information pieces to be permitted relative to one download request, a plurality of items of download permission data 65 having the same download request ID 641 are stored.

The download key data is created as a data string different for each download request. The download key data may be a character string created by converting by hash function from a character string including the medical institution ID of the requesting medical institution 2, the patient ID of the requested patient 1, and the request contents. The time limit for download 655 may be determined by the medical institution 2 on the medical information download request page 503, by the patient 1 on the medical information download permission page 504, or by the management server 30 based on a pre-set download period. However, when the patient 1 clicks an icon indicating “DO NOT PERMIT” on the medical information download permission page 504, the patient terminal 10 transmits information on download rejection to the management server 30. The management server 30 modifies a permission flag 646 in the corresponding reference request data 64 from “not permitted” to “rejected.”

When the operator of the medical institution 2 having received the download permission downloads the medical information, the support system performs download execution S102-3. First, the operator of the medical institution 2 transmits the download key data to the management server 30 (S102-3-1) using the medical institution terminal 20. Next, the management server 30 confirms whether or not the received download key data and the transmitting medical institution ID are present in the download permission data 65 and authenticates the download key data (S102-3-2). In addition, if the management server 30 confirms that the time limit for download has not expired, it transmits a viewer page of the corresponding medical information to the medical institution terminal 20 (S102-3-3). The medical institution terminal 2 thereby displays the viewer page of the medical information (S102-3-4). When transmitting the viewer page of the medical information, the management server 30 creates and stores download history data.

FIG. 14 is a view showing an example of the format for storing the download history data according to the embodiment of the present invention. The download history data 66 includes a download history ID 661, a download permission ID 651, a medical information ID 631, and a download date and time 662. When download is made a plurality of times relative to one download permission data, a plurality of items of download history data having the same download permission ID 651 are stored.

As is clear from the above description, according to the embodiment of the present invention, it is possible for a patient to determine when and by whom his or her medical information is downloaded and viewed, and which medical information is to be downloaded and viewed, so that the patient can safely control the information. In addition, a doctor of the medical institution can perform medical treatment based on consistent information by studying past medical information.

(Description of the Procedure of Medical Information Reference 103)

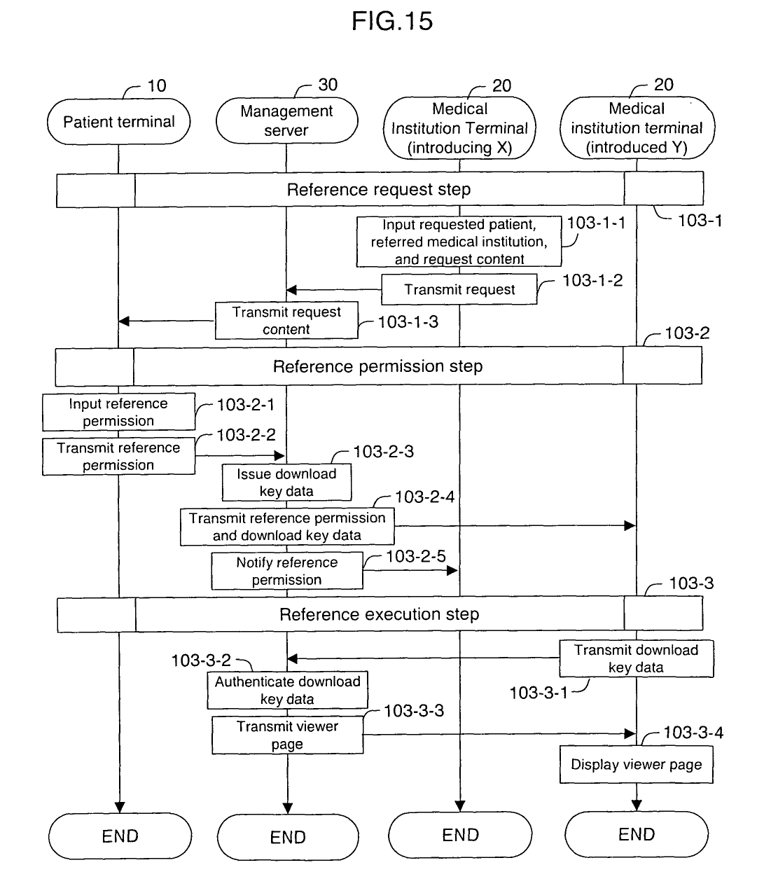

FIG. 15 is a flow chart showing an example of the procedure of medical information reference 103 according to the embodiment of the present invention. When an operator of a first medical institution (X) 2 requests the patient 1 to allow the medical institution (Y) 2 to download and view the past medical information 4 to refer a patient 1 to a second medical institution (Y) 2, the support system first performs reference request S103-1. The operator of the medical institution (X) 2 inputs the name of the requested patient 1, the referred medical institution (Y) 2, and request content on the terminal 20 of the medical institution (X) (S103-1-1), and transmits the request to the management server 30 (S103-1 -2).

On the medical information reference request page 503 shown as the example in FIG. 10, the operator of the medical institution (X) selects “for reference” in the item for download purpose and inputs the name of a medical institution to be introduced, thereby designating the medical institution (Y) 2. Next, the management server 30 having received the request transmits the request content to a patient terminal 10 used by the requested patient 1 (S103-1-3). At the same time, the management server 30 creates and stores download request data 64.

When the patient 1 having received the request permits reference of the medical information, the support system performs reference permission S103-2. First, the patient 1 confirms the content of the reference request and then inputs permission for the reference (S103-2-1) on the patient terminal 10. Then, the patient terminal 10 transmits the permission for the medical information reference to the management server 30 (S103-2-2).

The management server 30 having received the reference permission issues download key data necessary to download and view the medical information 4 (S103-2-3), and transmits the reference permission and download key data to the medical institution terminal 20 of the referred medical institution (Y) 2 (S103-2-4). Then, the management server 30 modifies a permission flag 646 in the corresponding reference request data 64 from “not permitted” to “permitted,” and then creates and stores download permission data 65 including the download key data.

The ID of the introduced medical institution (Y) 2 is set as the permitted medical institution ID 652 of the download permission data 65. Further, a reference permission notice is transmitted to the terminal 20 of the requesting medical institution (X) 2 (S103-2-5). The download key data is created as a different string of data for each reference request. It may be a character string created by converting by hash function from a character string including the medical institution ID of the requesting medical institution (X) 2, the patient ID of the requested patient 1, and the request content.

When the operator 2 of the medical institution (Y) having received the reference permission downloads the medical information, the support system performs reference execution S103-3. First, the operator of the medical institution (Y) 2 transmits the download key data from the medical institution terminal 20 to the management server 30 (S103-3-1). Next, the management server 30 confirms whether or not the received download key data and the transmitting medical institution ID are present in the download permission data 65, and authenticates the download key data (S103-3-2). In addition, if the management server 30 confirms that the time limit for download has not expired, it transmits a viewer page of corresponding medical information to the medical institution terminal 20 of the medical institution (Y) (S103-3-3). The medical institution terminal 20 of the medical institution (Y) 2 thereby displays the viewer page of the medical information that has been requested by the operator of the medical institution (X) 2 (S103-3-4). When transmitting the viewer page of the medical information, the management server 30 creates and stores download history data.

As is clear from the above description, the delivery and reception of information between medical institutions for referring a patient from one institution to another can be safely controlled by the patient according to the embodiment of the present invention. Since the patient reference can be smoothly conducted, the quality and efficiency of medical care in a region with limited medical care resources can be enhanced.

According to the medical support system of the present invention, the patient enjoys benefits in that he or she can efficiently receive high-quality medical care while controlling his or her own medical information securely. On the other hand, medical institutions can provide high-quality medical care while referring to past medical information from other medical institutions. The invention also prevents overlapping administration of drugs and allows medical treatment invoices from a plurality of medical institutions to be checked.

Further, in addition to the medical information described above, information concerning checkup examinations, health guidance, nursing care, or the like can be stored in the same manner as in the embodiments of the present invention. In this manner, it becomes possible to provide efficient and high-quality health guidance or medical care, based on individual information regarding medical care, health care, and welfare over lifetime.

The present invention can provide a medical support system which can store medical information on a patient disclosed by a plurality of medical institutions and allow the patient to manage the upload and download of the medical information.

All publications, patents and patent applications cited herein are incorporated herein by reference in their entirety.

What is claimed is:

1. A medical support system comprising:
patient terminals used individually by a plurality of patients;
medical institution terminals used individually by each of a plurality of medical institutions; and
a management server operated by a data management institution, which are connected to each other via a network, wherein
said management server comprises a medical information storage device for storing medical information of said patient including medical records, examination results, and medical images, and a medical information control device for controlling the input and output of said medical information into and from said medical information storage device,
wherein said medical information control device performs:
(1) an upload request step for, upon receiving a request from said patient terminal to upload said medical information of said patient managed by said medical institution in said management server, transmitting upload permission key data for uploading said medical information of said patient in said management server and a medical information upload request content to be uploaded by a patient's request to said medical institution terminal used by said requested medical institution;
(2) an upload standby step for, upon receiving said upload permission key data transmitted from said medical institution terminal, authenticating the validity of said upload permission key data, transmitting to said medical institution terminal a permission response for transmitting said medical information to said management server, and deferring said upload; and
(3) an upload execution step for, upon receiving said medical information transmitted from said medical institution terminal, storing said medical information in said medical information storage device.

2. A medical support system comprising:
patient terminals used individually by a plurality of patients;
medical institution terminals used individually by a plurality of medical institutions; and
a management server operated by a data management institution, which are connected to each other via a network, wherein
said management server comprises a medical information storage device for storing medical information of said patient including medical records, examination results, and medical images, and a medical information control device for controlling the input and output of said medical information into and from said medical information storage device,
wherein said medical information control device performs:
(1) a download request step for, upon receiving a medical information download request for download and view said medical information of said patient from said medical institution terminal used by said medical institution, transmitting a content of said medical information download request to said patient terminal used by said requested patient;
(2) a download permission step for, upon receiving a permission response for downloading said medical information from said patient terminal, transmitting download permission key data which permits said medical institution to download and view said medical information to said medical institution terminal; and
(3) a download execution step for, upon receiving said download permission key data transmitted from said medical institution terminal, authenticating the validity of said download permission key data and transmitting said medical information to said medical institution terminal.

3. A medical support system comprising:
patient terminals used individually by a plurality of patients;
medical institution terminals used individually by a plurality of medical institutions; and
a management server operated by a data management institution, which are connected to each other via a network, wherein
said management server comprises a medical information storage device for storing medical information of said patient including medical records, examination results, and medical images, and a medical information control device for controlling the input and output of said medical information into and from said medical information storage device,
wherein said medical information control device performs:
(1) a reference request step for, upon receiving a medical information reference request from a first medical institution terminal to allow a second medical institution, to which said patient is introduced, to download and view said medical information of said patient, transmitting a content of said medical information reference request to said patient terminal used by said requested patient;
(2) a reference permission step for, upon receiving a permission response from said patient terminal to permit said second medical institution to download and view said medical information, transmitting download permission key data to permit said second medical institution to download and view said medical information to said second medical institution terminal; and
(3) a reference execution step for, upon receiving said download permission key data transmitted from said second medical institution terminal, authenticating the validity of said download permission key data and transmitting said medical information to said second medical institution terminal.
